(19) United States
(12) Patent Application Publication
(10) Pub. No.: US 2014/0277181 A1
(43) Pub. Date: Sep. 18, 2014

(54) VARIABLE ANGLE LOCKING SCREW AND PLATE

(71) Applicant: Arthrex, Inc., Naples, FL (US)

(72) Inventor: Adam Garlock, Bonita Springs, FL (US)

(73) Assignee: Arthrex, Inc., Naples, FL (US)

(21) Appl. No.: 14/104,085

(22) Filed: Dec. 12, 2013

Related U.S. Application Data
(60) Provisional application No. 61/782,812, filed on Mar. 14, 2013.

(51) Int. Cl. A61B 17/80

(52) U.S. Cl. CPC A61B 17/8057
USPC 606/291

(57) ABSTRACT

A fastener and plate assembly includes a plate including a hole having a first threaded lobe with a first axis and a second threaded lobe with a second axis. The second axis is offset relative to the first axis. The assembly also includes a threaded fastener received in one of the first threaded lobe and the second threaded lobe of the hole to install the threaded fastener in one of a first orientation and a second orientation, respectively.

### REFERENCE TO RELATED APPLICATION

This application claims priority to U.S. Provisional Patent Application No. 61/782,812 filed Mar. 14, 2013.

### BACKGROUND OF THE INVENTION

During a surgical procedure, such as a bone fixation procedure that secures a fractured bone, a plate can be attached to bone with locking screws. The orientation of a locking screw is determined by a hole in the plate, resulting in only one orientation of the locking screw relative to the plate.

### SUMMARY OF THE INVENTION

A fastener and plate assembly includes a plate including a hole having a first threaded lobe with a first axis and a second threaded lobe with a second axis. The second axis is offset relative to the first axis. The assembly also includes a threaded fastener received in one of the first threaded lobe and the second threaded lobe of the hole to install the threaded fastener in one of a first orientation and a second orientation, respectively.

A fastener and plate assembly includes a plate including a hole having a first threaded lobe with a first axis, a second threaded lobe with a second axis, and a third threaded lobe with a third axis. The second axis is offset relative to the first axis, and the third axis is offset relative to the first axis. The assembly includes a threaded fastener received in one of the first threaded lobe, the second threaded lobe and the third threaded lobe of the hole. Threads of the threaded fastener engage threads of one of the first threaded lobe, the second threaded lobe or the third threaded lobe for at least 180° around a central axis of the threaded fastener to install the threaded fastener in one of a first orientation, a second orientation, and a third orientation, respectively.

A method of forming a hole in a plate includes the steps of forming a first base hole through a thickness of a plate and forming threads in the plate around the first base hole. The first base hole having a first axis. The method also includes the steps of forming a second base hole through the thickness of the plate that partially overlaps with the first base hole and forming threads in the plate around the second base hole. The second base hole has a second axis that is offset relative to the first axis. The method further includes the steps of receiving threads of a threaded fastener in one of the first base hole and the second base hole such that the threads of the threaded fastener engage the threads of the plate of one of the first base hole and the second base hole to install the threaded fastener in one of a first orientation and a second orientation, respectively.

These and other features of the present invention can be best understood from the following specification and drawings, the following of which is a brief description.

### BRIEF DESCRIPTION OF THE DRAWINGS

The various features and advantages of the invention will become apparent to those skilled in the art from the following detailed description of the currently preferred embodiment. The drawings that accompany the detailed description can be briefly described as follows:

FIG. 1 illustrates a plate including a plurality of threaded holes;

FIG. 2 illustrates the forming of the first base hole;

FIG. 3 illustrates the forming of the second base hole;

FIG. 4 illustrates the forming of the third base hole;

FIG. 5 illustrates a threaded hole of the plate of FIG. 1;

FIG. 6 illustrates a cross sectional view of the plate of FIG. 1 taken along the line 6-6;

FIG. 7 illustrates an enlarged view of a threaded hole of FIG. 6;

FIG. 8 illustrates a cross sectional view of the plate of FIG. 1 taken along the line 8-8;

FIG. 9 shows an enlarged view of a threaded hole of FIG. 8;

FIG. 10 illustrates a locking screw;

FIG. 11 illustrates the locking screw installed relative to a first axis;

FIG. 12 illustrates the locking screw installed relative to a second axis;

FIG. 13 illustrates the locking screw installed relative to a third axis; and

FIG. 14 illustrates several possible installation orientations of the locking screws.

### DETAILED DESCRIPTION OF THE PREFERRED EMBODIMENTS

FIG. 1 illustrates a plate 10 including a plurality of threaded holes 12. Although 8 threaded holes 12 are illustrated, the plate 10 can include any number of threaded holes 12. In one example, the plate 10 has a thickness T of approximately 3 mm. In one example, the plate 10 is made of carbon fiber or plastic. In another example, the plate 10 is made of metal.

As shown in FIG. 2, a first base hole 13 (shown by two substantially parallel solid lines) is formed in the plate 10. In one example, the first substantially circular base hole 13 is formed by drilling. The first substantially circular base hole 13 has a first longitudinal axis 18 that is substantially perpendicular to a plane defined by the plate 10.

As shown in FIG. 3, a second substantially circular base hole 20 (shown by two substantially parallel dashed lines) is then formed in the plate 10. In one example, the second substantially circular base hole 20 is formed by drilling. The second substantially circular base hole 20 has a second longitudinal axis 22 that is angled about X° from the first longitudinal axis 18. In one example, X° is about 0° to 20°. In one example, X° is about +12°. However, X can be any number.

As the first substantially circular base hole 13 and the second substantially circular base hole 20 partially overlap, a single hole including two lobes is formed. The lobes are formed by areas where the first substantially circular base hole 13 and the second substantially circular base hole 20 do not overlap. The spacing of the second substantially circular base hole 20 relative to the first substantially circular base hole 13 is shown exaggerated in FIG. 3 for illustrative purposes only.

As shown in FIG. 4, a third substantially circular base hole 24 (shown by two substantially parallel dotted lines) is then formed in the plate 10. In one example, the third substantially circular base hole 24 is formed by drilling. The third substantially circular base hole 24 has a third longitudinal axis 26 that is angled about Y° from the first longitudinal axis 18. In one example, Y° is about 0° to −20°. In one example, Y° is about −12°. However, Y can be any number.

As the hole with two lobes formed by the first substantially circular base hole 13 and the second substantially circular base hole 20 overlap with the third substantially circular base hole 24, a single hole with three lobes is formed. The lobes are formed by areas where the first substantially circular base hole 13, the second substantially circular base hole 20 and the third substantially circular base hole 24 do not overlap. The spacing of the third substantially circular base hole 24 relative to the first substantially circular base hole 13 is shown exaggerated in FIG. 4 for illustrative purposes only.

Each of the substantially circular base holes 13, 20 and 24 are then threaded to form the threaded hole 12, as shown in FIG. 5. In one example, each of the substantially circular base holes 13, 20 and 24 is threaded by a thread whirling process.

A threading instrument is inserted into the area created by the first substantially circular base hole 13 and aligned with the first longitudinal axis 18 to create a first thread 28 in the plate 10, as shown in FIGS. 6 and 7. The threading instrument is inserted into the area created by the second substantially circular base hole 20 and aligned with the second longitudinal axis 22 to create a second thread 30 in the plate 10, as shown in FIGS. 8 and 9. The threading instrument is inserted into the area created by the third substantially circular base hole 24 and aligned with the third longitudinal axis 26 to create a third thread 32 in the plate 10.

As shown in FIG. 10, a threaded fastener 34, such as a locking screw, includes a threaded head 36 and a shaft 38 with a plurality of threads 40. The threaded fastener 34 is received in the plate 10 and the bone 50 that is located under the plate 10 to secure the plate 10 to the bone 50. In the above-described example, the threaded fastener 34 can be inserted in one of three orientations that each correspond to one of the holes 13, 20 and 24 including the threads 28, 30 and 32, respectively. The threads 40 of the threaded fastener 34 engage one of the threads 28, 30 and 32 of one of the holes 13, 20 and 24, respectively, of the threaded hole 12 at least 180° around a central axis Q of the threaded fastener 34 to secure the plate 10 to the bone 50.

As shown in FIG. 11, when the threaded fastener 34 is installed along the first longitudinal axis 18, the threaded head 36 of the threaded fastener 34 engages the first thread 28 of the first hole 13 as shown in FIG. 7 to secure the threaded fastener 34 in the first orientation to secure the plate 10 to the bone 50.

As shown in FIG. 12, when the threaded fastener 34 is installed along the second longitudinal axis 22, the threaded head 36 of the threaded fastener 34 engages the second thread 30 of the second hole 20 as shown in FIG. 9 to secure the threaded fastener 34 in the second orientation to secure the plate 10 to the bone 50.

As shown in FIG. 13, when the threaded fastener 34 is installed along the third longitudinal axis 26, the threaded head 36 of the threaded fastener 34 engages the third thread 32 of the third hole 24 as shown in FIG. 9 to secure the threaded fastener 34 in the third orientation to secure the plate 10 to the bone 50.

FIG. 14 illustrates the three possible orientations of the threaded fastener 34 relative to the plate 10. The threaded fastener 34 can be installed in the plate 10 at variable angles along variable axes to secure the plate 10 to the bone 50. The threaded fastener 34 allows for multiple threads of engagement. Additionally, cross threading or thread deformation is not required to achieve the variable angle locking.

In the described example, the first longitudinal axis 18, the second longitudinal axis 22 and the third longitudinal axis 26 are all in the same plane. However, the first longitudinal axis 18, the second longitudinal axis 22 and the third longitudinal axis 26 can be in different planes and at different orientations relative to each other. This allows the threaded fastener 34 to be located at different orientations based on a patient's anatomy. Additionally, although a threaded hole 12 with three installation orientations is illustrated and described, the threaded hole 12 can include any number of lobes to define any number of possible orientations of the threaded fastener 34. For example, the threaded hole 12 can include two lobes to allow for two different orientations of the threaded hole 12.

The foregoing description is only exemplary of the principles of the invention. Many modifications and variations of the present invention are possible in light of the above teachings. The preferred embodiments of this invention have been disclosed, however, so that one of ordinary skill in the art would recognize that certain modifications would come within the scope of this invention. It is, therefore, to be understood that within the scope of the appended claims, the invention may be practiced otherwise than as specifically described. For that reason the following claims should be studied to determine the true scope and content of this invention.

What is claimed is:

1. A fastener and plate assembly comprising:
a plate including a hole having a first threaded lobe with a first axis and a second threaded lobe with a second axis, wherein the second axis is offset relative to the first axis; and
a threaded fastener received in one of the first threaded lobe and the second threaded lobe of the hole to install the threaded fastener in one of a first orientation and the second orientation, respectively.

2. The fastener and plate assembly as recited in claim 1 wherein the plate is made of one of carbon fiber, plastic or metal.

3. The fastener and plate assembly as recited in claim 1 wherein the first axis is substantially perpendicular to a plane defined by the plate.

4. The fastener and plate assembly as recited in claim 1 wherein an angle defined between the first axis and the second axis is about 0° to 20°.

5. The fastener and plate assembly as recited in claim 1 including a third threaded lobe with a third axis, wherein the third axis is offset relative to the first axis and the second axis.

6. The fastener and plate assembly as recited in claim 5 wherein an angle defined between the first axis and the third axis is about 0° to −20°.

7. The fastener and plate assembly as recited in claim 1 wherein threads of the threaded fastener engage threads of one of the first threaded lobe and the second threaded lobe for at least 180° around a central axis of the locking screw.

8. A fastener and plate assembly comprising:
a plate including a hole having a first threaded lobe with a first axis, a second threaded lobe with a second axis, a third threaded lobe with a third axis, wherein the second axis is offset relative to the first axis, and the third axis is offset relative to the first axis; and
a threaded fastener received in one of the first threaded lobe, the second threaded lobe and the third threaded lobe of the hole, wherein threads of the threaded fastener engage threads of one of the first threaded lobe, the second threaded lobe or the third threaded lobe for at least 180° around a central axis of the threaded fastener to install the threaded fastener in one of a first orientation, s second orientation, and a third orientation, respectively.

9. The fastener and plate assembly as recited in claim 8 wherein the plate is made of one of carbon fiber, plastic or metal.

10. The fastener and plate assembly as recited in claim 8 wherein the first axis is substantially perpendicular to a plane defined by the plate.

11. The fastener and plate assembly as recited in claim 8 wherein an angle defined between the first axis and the second axis is about 0° to 20°.

12. The fastener and plate assembly as recited in claim 8 wherein an angle defined between the first axis and the third axis is about 0° to −20°.

13. A method of forming a hole in a plate, the method comprising the steps of:
forming a first base hole through a thickness of a plate, the first base hole having a first axis;
forming a second base hole through the thickness of the plate that partially overlaps with the first base hole, wherein the second base hole has a second axis that is offset relative to the first axis;
forming threads in the plate around the first base hole;
forming threads in the plate around the second base hole; and
receiving a threaded fastener in one of the first base hole and the second base hole such that the threads of the threaded fastener engage the threads of the one of the first base hole and the second base hole to install the threaded fastener in one of a first orientation and a second orientation, respectively.

14. The method as recited in claim 13 wherein the step of forming the first base hole and the second base hole includes drilling.

15. The method as recited in claim 13 wherein the first axis is substantially perpendicular to a plane defined by the plate.

16. The method as recited in claim 13 including the step of forming a third base hole through the thickness of a plate that partially overlaps with the first base hole and the second base hole, wherein the third base hole has a third axis, and the third axis is offset relative to the first axis and the second axis.

17. The method as recited in claim 13 wherein the step of receiving the threaded fastener in the plate includes engaging threads of the threaded fastener with the threads of one of the first threaded lobe or the second threaded lobe for at least 180° around a central axis of the threaded fastener to install the threaded fastener in one of a first orientation and the second orientation, respectively.
